(19) United States
(12) Patent Application Publication
(10) Pub. No.: US 2010/0160766 A1
(43) Pub. Date: Jun. 24, 2010

(54) MAGNETIC RESONANCE METHOD AND SYSTEM FOR FLOW MEASUREMENT

(76) Inventors: Andreas Greiser, Erlangen (DE); Jens Guehring, Monmouth Junction, NJ (US); Mehmet Akif Guelsuen, Princeton, NJ (US); Arne Littmann, Erlangen (DE); Peter Schmitt, Weisendorf (DE); Michael Zenge, Nuernberg (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/629,375

(22) Filed: Dec. 2, 2009

(30) Foreign Application Priority Data
Dec. 2, 2008 (DE) 10 2008 060 050.4

(51) Int. Cl. A61B 5/055

(52) U.S. Cl. 600/419

(57) ABSTRACT

In a method for flow measurement with a magnetic resonance system and a correspondingly designed magnetic resonance system angiography measurement data of a volume are obtained within a body to be examined, a vessel is determined depending on a user input by means of the angiography measurement data, dimensions and an orientation of the vessel are automatically determined from the angiography measurement data, a slice geometry for the flow measurement is automatically determined depending on the dimensions and the orientation, and the flow measurement is implemented using the slice geometry.

### BACKGROUND OF THE INVENTION

1. Field of the Invention

The present invention concerns a method with which a flow measurement by means of a magnetic resonance system is implemented and a correspondingly designed magnetic resonance system.

2. Description of the Prior Art

For a two-dimensional phase contrast flow measurement with a magnetic resonance system, in addition to standard slice orientations, a slice that, within a vessel of interest, proceeds perpendicularly through said vessel or along this vessel, must be planned in order to implement the phase contrast flow measurement for this slice.

After the generation of measurement data of the phase contrast flow measurement, at present these measurement data are analyzed in a post-processing phase by means of a manual or semi-automatic segmentation in order to demarcate the vessels of interest in every temporal phase relative to a surrounding tissue, in order to be able to ultimately calculate relevant flow parameters for the vessels of interest as a result. This procedure (i.e. this measurement and evaluation workflow) is very complex and time-consuming—for example as a supplement to an angiography measurement—and therefore cannot be implemented within the scope of an angiography measurement due to the wait time for a patient.

A planning of a flow measurement (for example of a phase contrast flow measurement) presently accordingly ensues interactively by an operator.

### SUMMARY OF THE INVENTION

An object of the present invention is to provide a method and a device for flow measurement that simplify such flow measurements.

This object is achieved in accordance with the present invention by a method for flow measurement with a magnetic resonance system that includes the following steps. Measurement data are acquired by MR angiography in a volume within a body to be examined. A vessel within the volume is defined depending on specifications or inputs by means of the measurement data acquired from the angiogram. Dimensions and an orientation of the previously determined vessel are determined using the measurement data acquired from the angiogram. A slice geometry for the flow measurement is determined depending on these dimensions and on the orientation of the vessel determined beforehand. The flow measurement is finally implemented by using this slice geometry.

In summary, the method according to the invention integrates a quantitative MR phase contrast flow measurement into an MR angiography method or, respectively, an MR angiography workflow. In contrast to the offline evaluation of the flow measurement that is known from the prior art, a robust online calculation of the flow parameters (i.e. a calculation in the course of the angiogram) is therefore possible so that nothing stands in the way of a use of the method according to the invention in everyday clinical situations.

A slice geometry indicates the precise occupation (position and orientation) of one or more slices in space, i.e. within the volume or vessel to be measured.

In comparison to the prior art, the flow measurement (for example a phase contrast flow measurement) can be implemented significantly more easily via the method according to the invention, and therefore can be implemented more quickly and in a reproducible manner (i.e. two phase contrast flow measurements conducted on the same subject lead to nearly the same results). Moreover, the results of the flow measurement are more precise since the flow measurement is implemented with a slice which is automatically optimally adapted to the vessel in which the flow measurement is to be conducted.

The angiogram by means of which the measurement data are acquired to determine the vessel can on the one hand be a time-of-flight angiogram or a phase contrast angiogram. The angiogram can be a CE angiogram (contrast enhanced angiogram) or a Non-CE angiogram (an angiogram operating without contrast agent).

The dimensions and the orientation of the vessel are thereby advantageously determined by means of a segmentation algorithm. Vessel properties with which the slice geometry or the slice planning can be additionally improved can also be determined in this segmentation.

To determine the vessel in which the flow measurement is to be implemented, in particular the measurement data acquired via the angiogram are presented on a display in graphical form such that the vessels situated within the volume are thereby graphically represented in a form that is understandable to a person. It is thus possible for the vessel in which the flow measurement is to be implemented to be marked on the display (for example by a physician) in order to define the vessel for the subsequent method steps proceeding automatically.

By the marking with which the vessel to be examined is determined, a specific point of interest within the vessel can also be defined. Therefore it is advantageous when a slice to be planned for the flow measurement passes exactly through this point or comprises this point.

There are two possibilities for the determination of the slice geometry depending on the dimensions and the orientation of the vessel. In the first possibility, the slice to be determined for the flow measurement is arranged parallel to the flow in the vessel, and therefore in the direction of the flow in the vessel, so that the flow essentially runs along the slice. In the second possibility, the slice to be determined for the flow measurement is arranged perpendicular to the flow in the vessel and therefore perpendicular to the flow direction so that the flow essentially flows through the slice in the thickness direction of the slice. In both the first and the second possibilities the slice is thereby arranged for the most part such that a section surface between the slice and the vessel is optimally situated in the center of the slice.

In order to fashion the slice according to the first and second possibility, a cylindrical structure can be optimally adapted to the vessel to be examined, for example. Starting from this cylindrical structure, the slice is then arranged parallel to a central axis of the cylindrical structure in the first possibility while in the second possibility the slice is arranged parallel to the circular area (or perpendicular to the central axis).

In an embodiment according to the invention, specific properties (for example a diameter of the vessel and/or a curvature radius of the vessel) are determined starting from the measurement data acquired during the angiogram. These properties or additional information can then be added as boundary conditions for segmentation in a quantitative evaluation of the results of the flow measurement or in a quantitative evaluation of the flow parameters of the flow measurement.

In other words, these properties can improve the segmentation, i.e. the precise position of the vessel to be examined, and therefore the results of the flow measurement.

The results of the flow measurement can be superimposed on a graphical representation of the measurement data acquired by means of the angiogram. The results of the flow measurement thereby effectively indicate a flow speed and a flow direction for every measured spatial point within the vessel. The overlay of the results of the flow measurement can thereby ensue via a flow vector representation or via a corresponding coloration. In a flow vector representation, the flow at a point is represented by a vector whose direction corresponds to the flow direction at this point and whose length corresponds to the flow speed at this point. In the coloration, each point is colored depending on the flow speed prevailing at this point.

Via the present invention it is possible that the local vessel properties are calculated or determined via a segmentation algorithm simply by clicking on or marking a vessel in a projection view of an angio-data set. These local vessel properties can then serve to determine the slice geometry or slice planning and as boundary conditions for the segmentation of the automatic flow evaluation (evaluation of the results of the flow measurement).

The present invention also encompasses a magnetic resonance system for flow measurement. The magnetic resonance system has a control unit that controls a scanner (data acquisition unit) of the magnetic resonance system, a receiver device that receives signals acquired by the scanner, and a computer that evaluates the acquired signals to implement a flow measurement. The magnetic resonance system is designed to implement an angiography procedure in order to acquire angiography measurement data of a volume within a body to be examined. Moreover, the magnetic resonance system has an input unit that receives a user input. Depending on this user input, by means of the angiography measurement data the magnetic resonance system is able to define a vessel. The dimensions and the orientation of this vessel are determined from the angiography measurement data of the computer. Depending on these dimensions and the orientation of the vessel, the magnetic resonance system generates or plans a slice geometry (i.e. in particular a slice) for the flow measurement. The magnetic resonance system then is able to implement the flow measurement by means of this slice geometry.

Moreover, the present invention also encompasses a computer-readable medium that can be loaded into a memory of a programmable controller or a computer of a magnetic resonance system. All or various embodiments of the method according to the invention that are described in the preceding can be executed by programming instructions with which the medium is encoded. The medium requires program components (for example libraries and auxiliary functions) in order to realize the corresponding embodiments of the method.

The computer-readable data medium has (for example a DVD, a magnetic tape or a USB stick electronically readable control information—in particular software stored therein. When this control information (software) is read from the data medium and is stored in a controller or computer of the magnetic resonance system, all embodiments according to the invention of the method described in the preceding can be implemented.

The present invention is particularly suitable to implement a phase contrast flow measurement within an angiography workflow by means of a magnetic resonance system. Naturally, the present invention is not limited to this preferred application field but rather can also be used for other types of a flow measurement by means of a magnetic resonance system, for example.

### BRIEF DESCRIPTION OF THE DRAWINGS

FIG. 1 graphically represents angiography measurement data.

FIG. 2 shows the definition of a vessel according to the invention using a marking.

FIG. 3 shows a slice fashioned according to the invention for the implementation of a flow measurement.

FIG. 4 shows measurement data of a phase contrast flow measurement according to the invention.

FIG. 5 shows numerical flow parameters for important regions within a slice superimposed on a graphical representation of this slice.

FIG. 6 shows a flow speed over time for a specific region.

FIG. 7 shows a numerical representation of specific flow information for a specific region within the slice.

FIG. 8 schematically shows a magnetic resonance system according to the invention.

### DESCRIPTION OF THE PREFERRED EMBODIMENTS

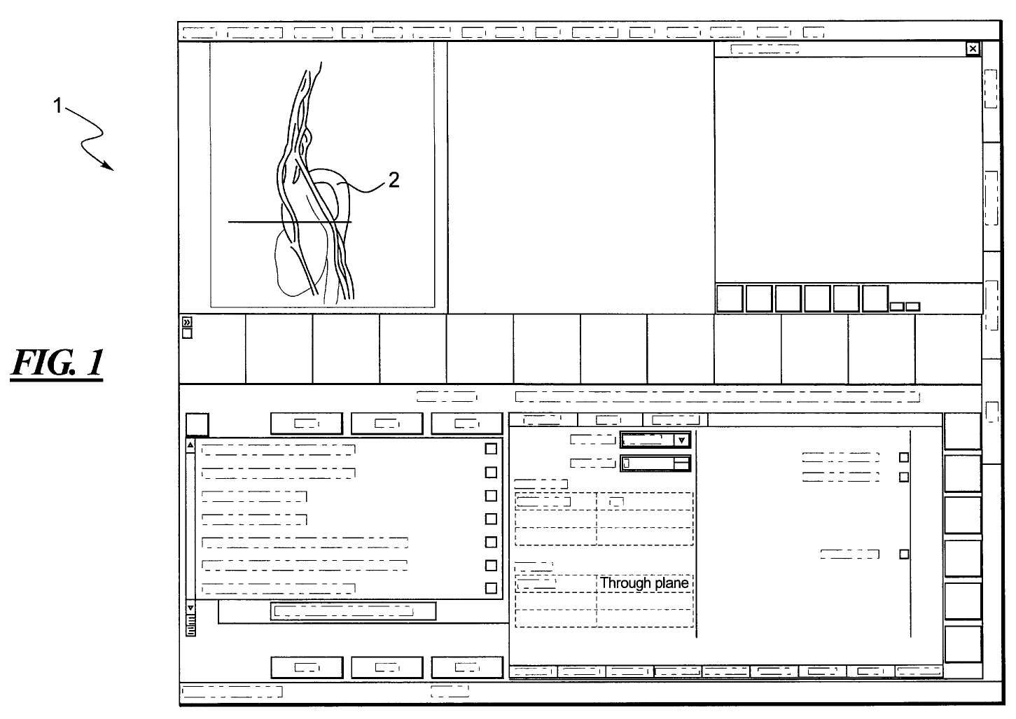

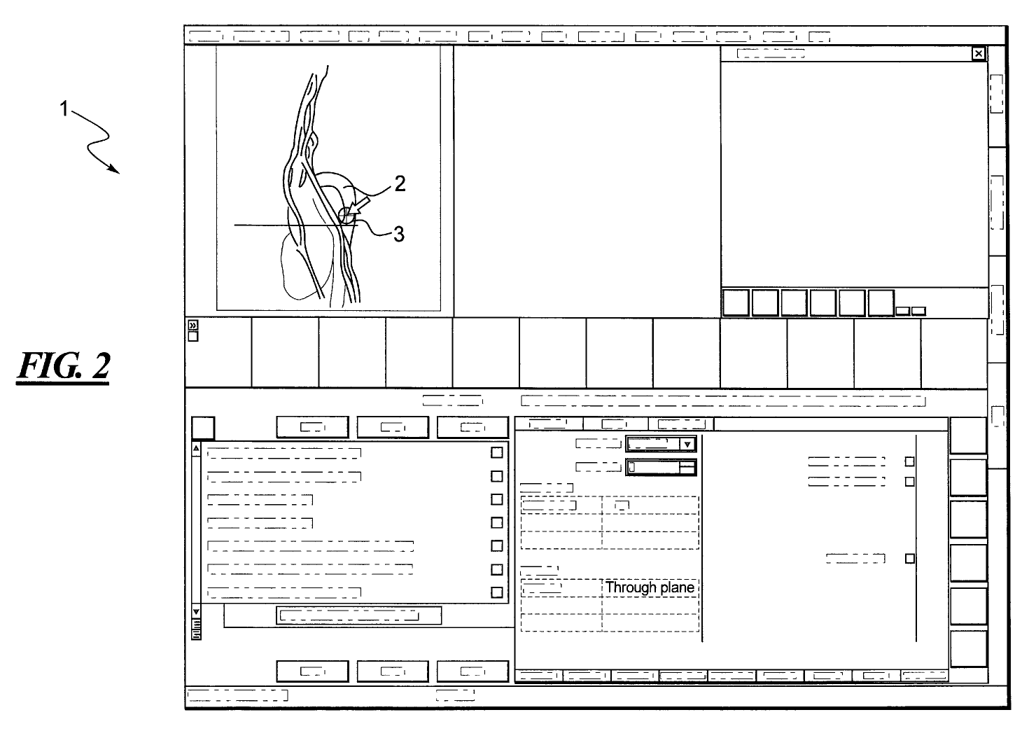

In the following the essential steps of a method according to the invention for flow measurement are presented using FIGS. 1 through 7.

First, angiography measurement data are generated as a reference and graphically presented, as is shown at the top left in a display 1 in FIG. 1.

For example, in the shown case a physician then marks with a cross-shaped marking 3 that vessel 2 in which a flow measurement should ultimately be conducted. With this marking 3 a spatial point is also defined through which a slice 4 has to run for the flow measurement.

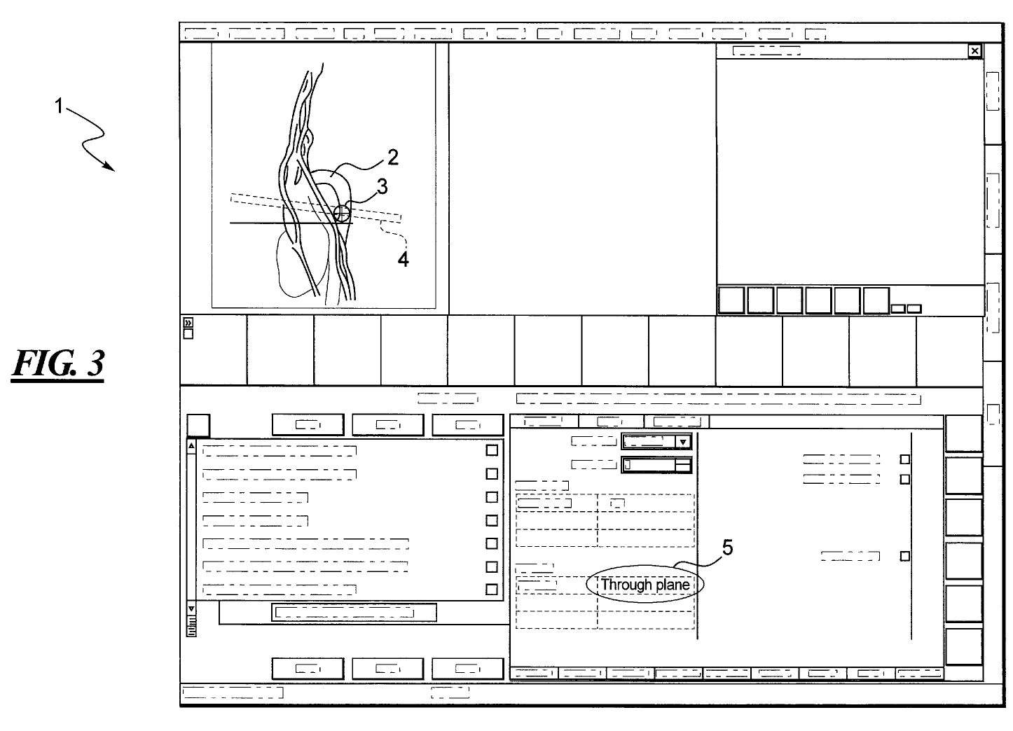

Starting from this marking 3, an orientation and a position of a slice 4 are calculated. For this a segmentation algorithm determines the precise bearing or the marked or defined vessel 2 in space. Depending on the bearing or orientation and position of the vessel 2, the orientation and position of the slice 4 are then determined from this. The slice 4 is thereby for the most part arranged either perpendicular or parallel to the vessel 2 or to the flow direction within the vessel 2. In the case shown in FIG. 3, the slice 4 is fashioned perpendicular to the vessel 2 and traverses the center point of the marking 3.

How the slice 4 is to be arranged in relation to the marked vessel 2 can be predetermined by the user by the specification of the direction, in this case “Through Plane” 5.

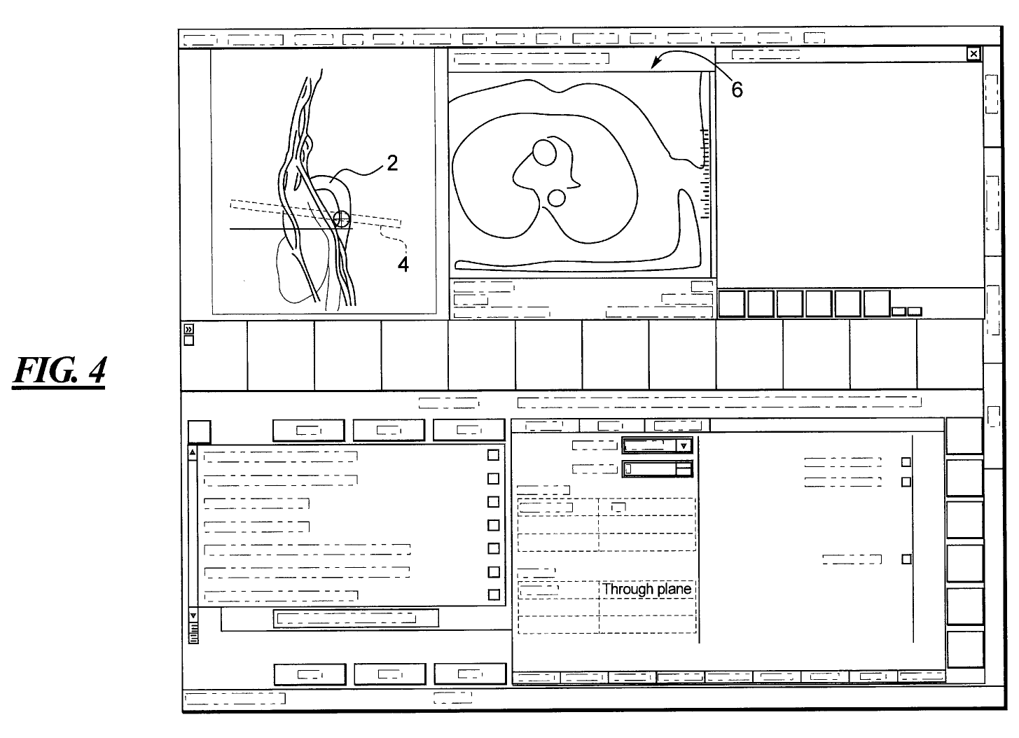

In a next step, a phase contrast flow measurement is conducted for the slice 4 planned beforehand. A graphically presented result of the results acquired via the phase contrast flow measurement is depicted in a display 6 at the right, next to the angio-presentation 1 in FIG. 4.

The measurement data acquired by means of the phase contrast flow measurement are analyzed inline or online (i.e. during the method and not within the framework of a post-processing) and corresponding flow parameters are generated which are shown at the right on a display 7 (next to the display 6 of the phase contrast flow measurement).

In other words, the results of the flow measurement can be acquired, evaluated and presented within the framework of an angiogram while the patient is located within the tomograph of the magnetic resonance system.

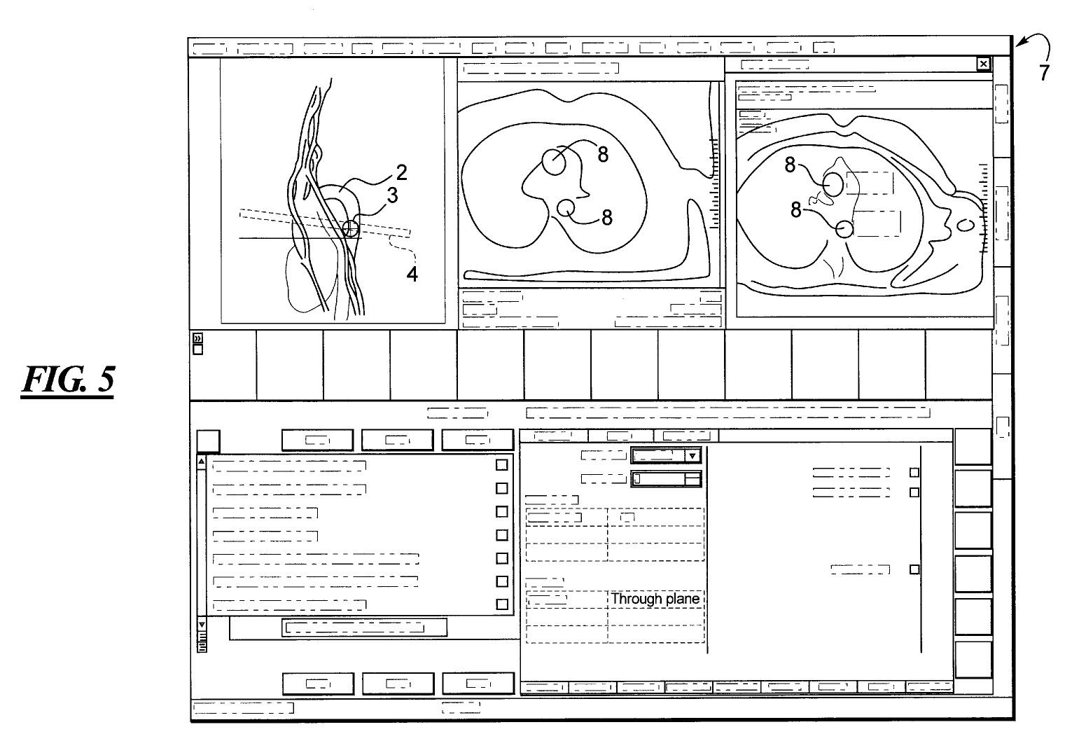

In the presentation in FIG. 5, the flow information for important regions within the slice 4 are represented in numerical form. For this regions within the slice 4 in which a significantly similar flow speed is present are determined by means of a segmentation algorithm which can also access results of the angiogram conducted beforehand. In FIG. 5 the two regions marked with the reference characters 8 are thereby regions which are also to be recognizable in the phase contrast image 6.

The respective following measurement results are presented in numerical form, spatially next to these regions 8 shown in the display 7:

the average flow speed within the corresponding region 8 (in cm/s)

the peak flow speed within the corresponding region 8 (in cm/s)

the fluid throughput in the corresponding region 8 (in ml/s)

an area which is taken up by the respective region 8 (in cm²)

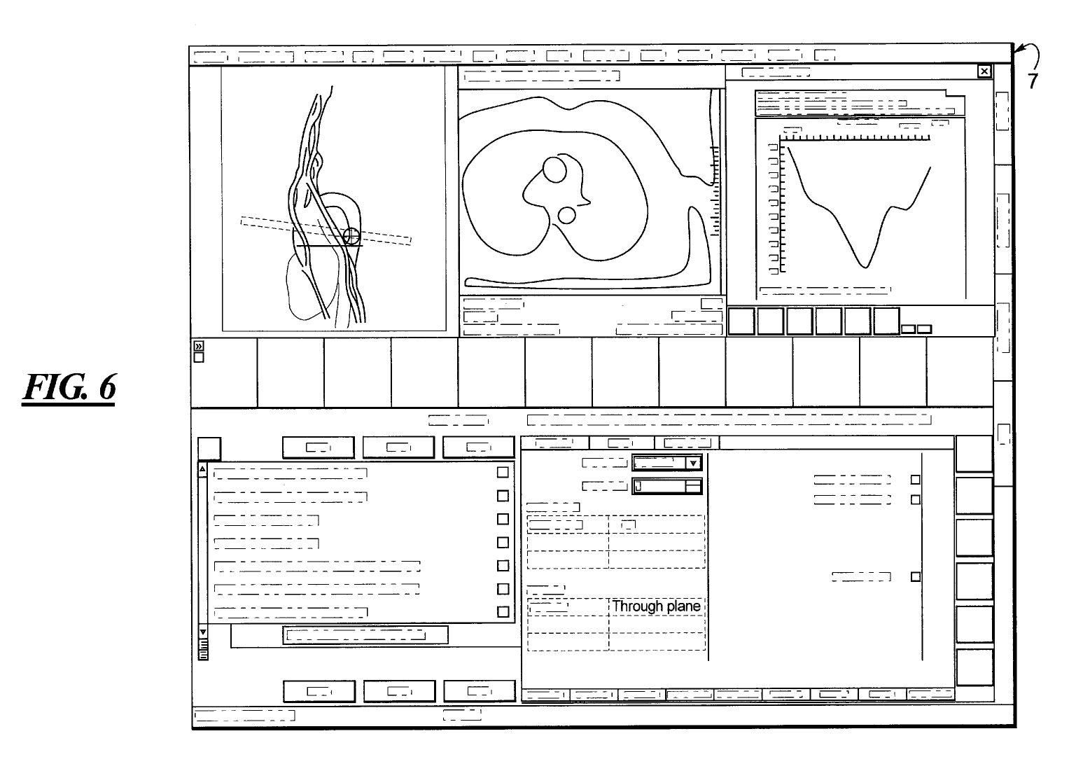

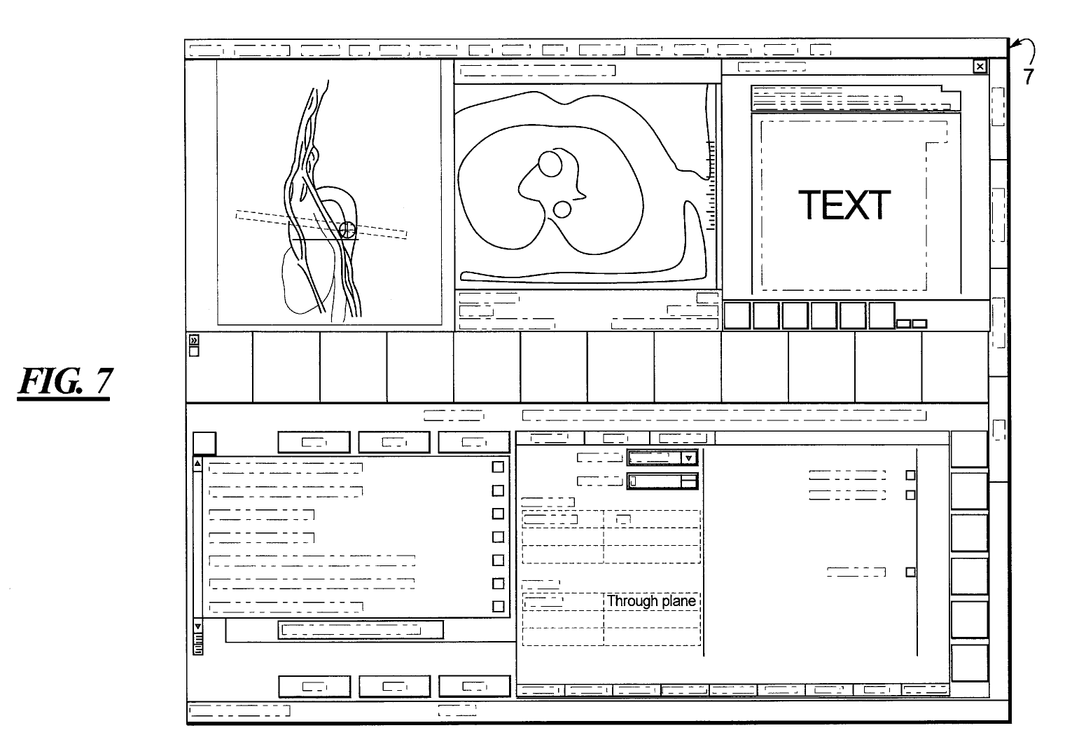

Additional possibilities for a presentation of the results of the phase contrast flow measurement are shown in FIG. 6 and FIG. 7. A flow speed over time for a specific region in the slice 4 is thereby shown in the display 7 of FIG. 6 while specific flow information (speed, flow quantity per time, area) is indicated in the display 7 of FIG. 7.

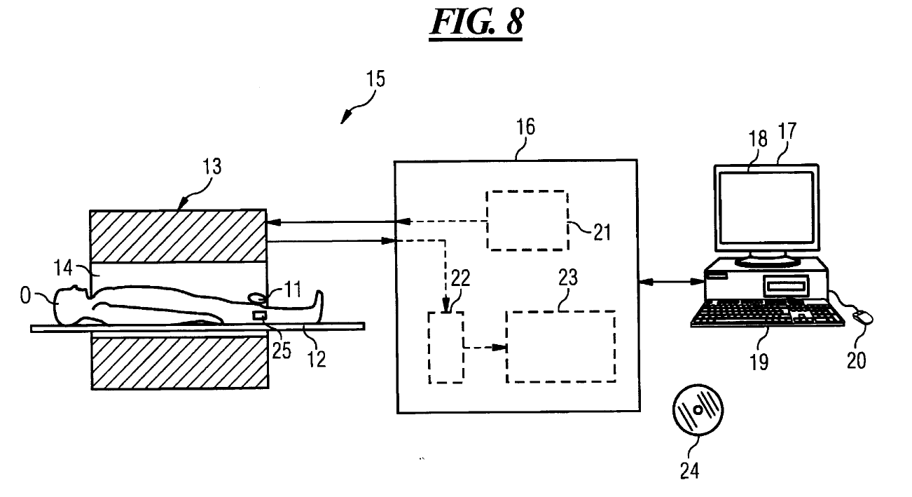

A magnetic resonance system 15 according to the invention is schematically shown in FIG. 8. The magnetic resonance system 15 essentially comprises a tomograph 13 with which the magnetic field necessary for the MR examination is generated in a measurement space 14, a table 12, a control device 16 with which the tomograph 13 is controlled and MR data from the tomograph 3 are acquired, and a terminal 17 connected to the control device 16.

The control device 16 includes a control unit 21, an acquisition unit 22 and a computer 23. During an MR examination (for example an angiogram or a flow measurement), MR data are acquired by the acquisition unit 22 means of the tomograph 13, wherein the tomograph 13 is controlled by the control unit 21 such that angiography measurement data are acquired in an angiogram in a measurement volume 25 which is located inside the body of a patient O lying on the table 12.

The computer 13 then prepares the angiography measurement data and measurement data of the flow measurement such that they can be graphically presented on a monitor 18 of the terminal 17. In addition to the graphical presentation of the angiography measurement data and measurement data of the flow measurement, a vessel 2 can be provided with a marking 4 and further specifications for the implementation of the angiogram and the flow measurement can be made by a user with the terminal 17 which, in addition to the monitor 18, comprises a keyboard 19 and a mouse 20. The software for the control device 16 can also be loaded via the terminal 17 into said control device 16, in particular into the computer 23. This software of the control device 16 implements the method according to the invention for flow measurement and can likewise be stored on a DVD 24 so that this software can then be read from the DVD 24 by the terminal 17 and be copied to the control device 16.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for magnetic resonance flow measurement in a magnetic resonance system, comprising the steps of:
in a magnetic resonance system, obtaining magnetic resonance angiography data from a volume within an examination subject;
supplying said angiography measurement data to a processor and providing a user input to said processor and, in said processor, determining a vessel in said volume from said angiography measurement data, dependent on said user input;
in said processor, automatically determining dimensions and an orientation of said vessel from said angiography measurement data;
in said processor, automatically determining a slice for implementing a flow measurement therein dependent on said dimensions and said orientation of said vessel; and
implementing said flow measurement in said slice using said coding with said magnetic resonance system.

2. A method as claimed in claim 1 comprising determining said dimensions and orientation of said vessel by image segmentation.

3. A method as claimed in claim 1 comprising defining said vessel by graphically presenting said angiography measurement data at a display wherein said vessel within said volume is graphically depicted, and providing said vessel with a marking.

4. A method as claimed in claim 3 comprising employing a point as said marking and defining said slice as a slice that contains said point.

5. A method as claimed in claim 1 comprising automatically determining a slice geometry for said slice that causes said slice to be substantially parallel to or substantially perpendicular to flow in said vessel.

6. A method as claimed in claim 1 comprising, in said processor, automatically determining properties of said vessel from said angiography measurement data and using said properties for evaluation of said flow measurement for segmentation.

7. A method as claimed in claim 6, comprising selecting said properties from the group consisting of a diameter of said vessel and a curvature radius of said vessel.

8. A method as claimed in claim 1 comprising superimposing results of said flow measurement on a graphical representation of said angiography measurement data.

9. A method as claimed in claim 8 comprising graphically depicting said angiography measurement data to show results of said flow measurement by a flow vector representation or color coding.

10. A method as claimed in claim 1 comprising implementing said flow measurement as a phase contrast flow measurement.

11. A magnetic resonance system for magnetic resonance flow measurement, comprising:
a magnetic resonance data acquisition unit;
a control unit that operates said magnetic resonance data acquisition unit to obtain magnetic resonance angiography data from a volume within an examination subject;
said control unit having an input supplied with said angiography measurement data and a user input and said control unit being configured to determine a vessel in said volume from said angiography measurement data, dependent on said user input;
said control unit being configured to automatically determine dimensions and an orientation of said vessel from said angiography measurement data;
said control unit being configured to automatically determine a slice for implementing a flow measurement therein dependent on said dimensions and said orientation of said vessel; and
said control unit being configured to operate said magnetic resonance data acquisition unit to implement said flow measurement in said slice using said coding.

12. A magnetic resonance system as claimed in claim 11 wherein said control unit being configured to determine said dimensions and orientation of said vessel by image segmentation.

13. A magnetic resonance system as claimed in claim 11 wherein said control unit being configured to define said vessel by graphically presenting said angiography measurement data at a display wherein said vessel within said volume is graphically depicted, and providing said vessel with a marking.

14. A magnetic resonance system as claimed in claim 13 wherein said control unit being configured to employ a point as said marking and defining said slice as a slice that contains said point.

15. A magnetic resonance system as claimed in claim 13 wherein said control unit being configured to automatically determine a slice geometry for said slice that causes said slice to be substantially parallel to or substantially perpendicular to flow in said vessel.

16. A magnetic resonance system as claimed in claim 11 said control unit being configured to automatically determine properties of said vessel from said angiography measurement data and using said properties for evaluation of said flow measurement for segmentation.

17. A magnetic resonance system as claimed in claim 16 wherein said control unit being configured to select said properties from the group consisting of a diameter of said vessel and a curvature radius of said vessel.

18. A magnetic resonance system as claimed in claim 16 wherein said control unit being configured to superimpose results of said flow measurement on a graphical representation of said angiography measurement data at a display unit connected to said control unit.

19. A magnetic resonance system as claimed in claim 18 wherein said control unit being configured to graphically depict said angiography measurement data to show results of said flow measurement by a flow vector representation or color coding.

20. A magnetic resonance system as claimed in claim 18 wherein said control unit being configured to operate said magnetic resonance data acquisition unit to implement said flow measurement as a phase contrast flow measurement.

21. A computer-readable medium encoded with programming instructions, said medium being loadable into a computerized control system of a magnetic resonance system and said programming instructions causing said control system to:
operate said magnetic resonance system to obtain magnetic resonance angiography data from a volume within an examination subject;
receive said angiography measurement data and a user input, and to determine a vessel in said volume from said angiography measurement data, dependent on said user input;
automatically determine dimensions and an orientation of said vessel from said angiography measurement data;
automatically determine a slice for implementing a flow measurement therein dependent on said dimensions and said orientation of said vessel; and
operate said magnetic resonance system to implement said flow measurement in said slice using said coding.
